(19) United States
(12) Patent Application Publication
(10) Pub. No.: US 2003/0143605 A1
(43) Pub. Date: Jul. 31, 2003

(54) Methods for the selection and cloning of nucleic acid molecules free of unwanted nucleotide sequence alterations

(76) Inventors: Si Lok, Seattle, WA (US); Stacey Tannheimer, Seattle, WA (US)

Correspondence Address: James M. Bogden, Attorney for Applicants, ZymoGenetics, Inc., 1201 Eastlake Avenue East, Seattle, WA, 98102, US

(21) Appl. No.: 10/308,925

(22) Filed: Dec. 3, 2002

Related U.S. Application Data
(60) Provisional application No. 60/336,888, filed on Dec. 3, 2001.

(51) Int. Cl. C12Q 1/68; C12P 19/34

(52) U.S. Cl. 435/6; 435/91.2

(57) ABSTRACT

The accurate synthesis of nucleic acid molecules is important for use of amplified nucleic acid molecules as hybridization probes, in the regulation of gene expression, as templates for the production of recombinant proteins, as diagnostic probes, and in forensic analyses. Methods are provided to separate nucleic acid molecules that are free of mutations from a population of nucleic acid molecules that contain unwanted nucleotide alternations.

### CROSS-REFERENCE TO RELATED APPLICATIONS

This application claims the benefit of U.S. provisional application No. 60/336,888 (filed Dec. 3, 2001), the contents of which are incorporated by reference.

### TECHNICAL FIELD

The present invention relates to methods, kits, and compositions suitable for efficient selection, isolation, and cloning of nucleic acid molecules that are free of unwanted nucleotide alterations.

### BACKGROUND OF THE INVENTION

Accurate synthesis of DNA is important for the use of the synthesized DNA molecules as hybridization probes, the regulation of gene expression, templates for the production of recombinant proteins, as well as for genetic or forensic analyses. Two important methods for the production of DNA are nucleic acid amplification methods, such as the polymerase chain reaction (PCR), and direct chemical synthesis. While widely used, these methods of synthesis are hampered by inherent errors, which compromise their application where accuracy is important.

A major cause of errors generated by PCR amplification is due to a misincorporation of nucleotides by DNA polymerase. This problem is particularly pronounced with polymerases lacking proof-reading activity. For example, Taq DNA polymerase has a reported base substitution error rate on the order of one error per 10,000 to 100,000 nucleotides polymerized under amplification conditions (Keohavong and Thilly, Proc. Nat'l Acad. Sci. USA 86:9253 (1989); Saili et al., Science 239:487 (1988); Eckert and Kunkel, Nucl. Acids Res. 18:3739 (1990)). The magnitude of such an error rate may result in an 80% probability for the occurrence of a mutation within a given 100 base pair amplicon after a twenty cycle amplification (Keohavong and Thilly, Proc. Nat'l Acad. Sci. USA 86:9253 (1989)). Thermostable Pfu, Vent, and other proof-reading polymerases, only offer a partial solution to the problem. These polymerases typically offer a two- to ten-fold increase in fidelity rate relative to Taq polymerase (Cline et al., Nucl. Acids Res. 24:3546 (1996)). Since the probability of nucleotide misincorporation per cycle is directly proportional to the size of the amplicon, polymerase-induced mutations, even with proof-reading polymerases, remain a significant problem in applications where the amplicon is large or when extensive number of PCR cycles are required. In addition to nucleotide misincorporation, other errors encountered during DNA amplification include: sequence deletions, sequence insertions, sequence rearrangements, chimeric formation, and heteroduplex formation between related amplicons. The impact of polymerase-induced errors relating to PCR applications in the area of genetic and forensic diagnostics and for cDNA cloning has been reported in the literature (Reiss et al, Nucl. Acids Res. 18:973 (1990); Smith and Modrich, Proc. Nat'l Acad. Sci. USA 93:4374 (1996); Ennis et al., Proc. Nat'l Acad. Sci. USA 87:2833 (1990)).

Many methods initially developed to analyze genetic changes in DNA have been adapted to identify polymerase-produced mutations in PCR. These methods are based on the formation of heteroduplexes after the completion of PCR. The denaturation and subsequent re-annealing of a population of PCR products, which contain a small mutant fraction, results in the formation heteroduplexes comprising a mutant strand hybridizing with a wild type strand or a mutant strand hybridizing with another mutant strand bearing different mutations. These heteroduplexes are characterized by the presence of one or more single-stranded regions representing nucleotide mismatches arising from either the occurrence of nucleotide misincorporations, deletion or insertion of nucleotides, or sequence rearangements occurring during DNA amplification. By contrast, the homoduplex fraction, which is double-stranded along their entire length, comprised predominantly non-mutated species.

Initially, the formation of DNA heteroduplexes and their subsequent interrogation were confined to diagnostic applications, and the technique was generally referred to as “heteroduplex mapping” (see, for example, Turner et al., Diabetes 44:1 (1995)). Later, investigators adopted the use of heteroduplex formation to remove PCR generated mutations or artifacts from an amplicon population. For example, Wagner used an immobilized DNA mismatch-binding protein, MutS, to bind heteroduplex DNA, thereby removing error-containing molecules from the PCR-amplified DNA samples by affinity chromatography (Wagner, U.S. Pat. No. 6,114,115 (2000); Wagner, U.S. Pat. No. 6,120,992 (2000)). Modrich and Smith further exploited Escherichia coli methyl-directed mismatch repair pathway to eliminate PCR-generated mutations (Smith and Modrich, Proc. Nat'l Acad. Sci. USA 94:6847 (1997); Modrich et al., U.S. Pat. No. 5,922,539 (1999)). By adding MutH and MutL to MutS, an active protein complex was produced, which not only recognized but also cleaved DNA duplexes containing base-pair mismatches (Smith and Modrich, Proc. Nat'l Acad. Sci. USA 94:6847 (1997); Modrich et al., U.S. Pat. No. 5,922,539 (1999)). The cleaved mutant products were then separated from the uncleaved and predominately non-mutated fraction by either gel electrophoresis or by HPLC prior to use. However, approaches based on the ability of MutS to bind mismatched DNA have limitations. Since cytosine-cytosine mismatches are not recognized by MutS, nucleotide misincorporation during PCR amplification, which later give rise to the formation of heteroduplexes containing cytosine-cytosine mismatches were not cleaved or eliminated from the amplicon population (Su et al., J. Biol.Chem. 263:68,299 (1988); Lahue et al., Science 245:160 (1989)). Moreover, the natural substrate for the MutH endonuclease subunit is DNA containing hemi-methylated d(GATC) sequences. Unmethylated d(GATC) sequences, such as those found in PCR products, are not cleaved efficiently and were subject to cleavage only if the reactions were allowed to progress for an extended periods of time, thereby further limiting the general usefulness of this approach (Au et al., J. Biol.Chem. 267:12142 (1992)).

There are number of other enzymes known to act on single-strand DNA, which can cleave mismatch bases within a heteroduplex with varying degree of efficiency and specificity. These include: S1 nuclease (Lundin et al., Nucl. Acids Res. 25:2535 (1997); Howard et al., BioTechniques 27:18 (1999)), Mung Bean endonuclease (Jaraczewski and Jahn, Genes Dev 7:95 (1993)), T4 endonuclease VII (T4E7) (Inganas et al., Clin. Chem. 46:1562 (2000)), T7 endonuclease I (T7EI) (Marshal et al., Nat. Genet. 9:177 (1995)), SP nuclease of spinach (Oleykowski et al., Biochemistry 38:2200 (1999)), and CEL I of celery (Oleykowski et al., Nucl. Acids Res. 26:4597 (1998); Yang et al., Biochemistry 39:3533 (2000)). In general, these enzymes have been employed to diagnosis genetic mutations in DNA. Lowell and Klein, BioTechniques 28:676-681 (2000), and Qiu et al., Applied and Environ. Microbiol. 67:880 (2001), reported the use of T7EI to digest heteroduplexes in PCR products generated when a mixture of homologous genes was used as PCR template. However, neither they nor any other investigators have describe the use of T7EI or other single-strand specific DNA nucleases for eliminating polymerase mediated nucleotide misincorportations, mutations, or other artifacts arising during reverse-transcription or during DNA amplification by PCR.

Another widely used method for cleaving mismatched DNA is Chemical Mismatch Cleavage (CMC). CMC was originally developed as a modification of the Maxium-Gilbert DNA sequence method, and the method was used for genetic analysis (Cotton et al., Mutation Research 285:125 (1993)). Mismatched thymine and cytosine nucleotides present in heteroduplex DNA were susceptible to modifications by osmium tetroxide and hydroxylamide, respectively (Cotton et al., Mutation Research 285:125 (1993)). Following modification of the mismatched bases by these chemical agents, heteroduplex DNA was cleaved at the modified nucleotides by hot piperidine and the resulting products were separated and analyzed by denaturing gel electrophoresis or by capillary electrophoresis (Ren et al, Clinical Chemistry 44:2108 (1998)). Later improvements to the basic CMC protocol included the replacement of toxic osmium tetroxide with potassium permanganate and tetraethylammonium chloride (Roberts et al Nucl. Acids Res. 25:3377 (1997); Lambrinakos et al., Nucl. Acids Res. 27:1866 (1999)). Along with the increased sensitivity for thymine mismatches relative to the use of osmium tetroxide, potassium permanganate also exhibited significant reactivity for mismatched guanine, cytosine, and adenine nucleotides, thereby increasing the usefulness of this reagent for genetic analysis (Rubin and Schmid, Nucl. Acids Res. 8:4613 (1980); Gogos et al., Nucl. Acids Res. 18:6807 (1990); Lambrinakos et al., Nucl. Acids Res. 27:1866 (1999)).

CMC has been shown to be a robust procedure and genetic mutations in a number of genes have been discovered using this method (Ren et al, Clinical Chemistry 44:2108 (1998)). However, the requirement to carry out the cleavage reaction at the modified bases with piperdine at 95° C., pH 12, has resulted in the denaturation of the homoduplex DNA strands. DNA denaturation during piperdine cleavage has precluded use in applications, such as cloning or transformation, where double-stranded DNA is required. Consequently, there have been no reports indicating the use of CMC as a method for eliminating heteroduplex DNA to rid mutations or artifacts arising during reverse-transcription or during DNA amplification by PCR.

A need still exists for a method to remove DNA molecules bearing unwanted nucleotide alterations from a mixture of amplified DNA. The selected DNA products of this method would be useful in a variety of applications, including genetic diagnosis or as a DNA template for the production of recombinant protein, and other applications where products from high fidelity sequence amplification are important.

### BRIEF SUMMARY OF THE INVENTION

The present invention provides methods, kits, and compositions suitable for efficient selection, isolation, and cloning of nucleic acid molecule fragments that are free of unwanted nucleotide alterations from a nucleic acid molecule population containing altered sequences. For example, DNA fragments subject to the invention include those amplified by PCR or other nucleic amplification methods, as well as DNA in whole or in part assembled from chemically synthesized oligonucleotides. As an illustration, sequence alterations or mutations may be due to errors introduced by RNA-dependent DNA polymerase (reverse transcriptase) during first strand cDNA synthesis, DNA polymerase used in second strand cDNA syntheses, polymerases employed in PCR, or other enzymes employed in methods of nucleic acid amplification. Unintended sequence alternations may also arise through failures in chemical oligonucleotide synthesis, or during the assembly of oligonucleotides to yield an intended product. Mutations or alterations in DNA include: single base substitutions, sequence insertions, sequence inversions, sequence deletions, sequence rearrangements, or chimerism.

### DESCRIPTION OF THE INVENTION

1. Overview

The methods described herein provide a means to separate nucleic acid molecules that are free of mutations from a population of nucleic acid molecules comprising a fraction containing unwanted nucleotide alternations. In brief, a population of double-stranded nucleic acid molecules is denatured, and the stands are allowed to re-anneal to yield populations of hetero- and homoduplexes. The heteroduplex population comprises mutant strands hybridizing with wild-type strands, or mutant strands hybridizing with other mutant strands bearing different mutations. The resulting heteroduplexes are characterized by the presence of one or more single-stranded regions representing areas of nucleotide mismatch. By contrast, the homoduplex population is double-stranded, and comprised predominantly of non-mutated species.

Standard nucleic acid molecule amplification procedures provide a means to synthesize nucleic acid molecules that comprise a target nucleotide sequence, or the complement of a target nucleotide sequence. This original nucleic acid molecule population will include amplified nucleic acid molecules that are mutated, compared with the nucleic acid molecule template used to produce the amplified nucleic acid molecules. Using the methods described below, one can produce a nucleic acid molecule population that is enriched for nucleic acid molecules comprising a target nucleotide sequence (or the complement of a target nucleotide sequence), compared with the original nucleic acid molecule population obtained by amplification.

In particular, the present invention provides methods for eliminating an amplified nucleic acid molecule having a nucleotide sequence that is mutated, compared with the target nucleotide sequence used to produce the amplified nucleic acid molecule, comprising: (a) obtaining a mixture of duplexes of amplified nucleic acid molecules, wherein the duplex mixture comprises homoduplexes and heteroduplexes, (b) treating the duplex mixture with a single-strand specific nuclease that cleaves a mismatched site within a heteroduplex, and (c) subcloning the nuclease-treated duplex mixture, wherein a cleaved duplex is unsuitable for subcloning. For example, a nucleic acid molecule that comprises subcloning restriction sites or recombination sites for in vitro recombination at both ends will be rendered unsuitable for subcloning after the nucleic acid molecule is cleaved.

The present invention also provides methods for eliminating an amplified nucleic acid molecule having a nucleotide sequence that is mutated, compared with the target nucleotide sequence used to produce the amplified nucleic acid molecule, comprising: (a) obtaining a mixture of duplexes of the amplified DNA molecules, wherein the duplex mixture comprises homoduplexes and heteroduplexes, (b) cleaving the duplex mixture with a single-strand specific nuclease that cleaves a mismatched site within a heteroduplex, wherein the cleavage produces an exposed 3′OH moiety, (c) treating the cleaved duplex mixture with a DNA polymerase and nucleotide analog conjugated with an affinity label to synthesize DNA that comprises the affinity label, and (d) incubating the treated duplex mixture of (c) with an affinity label capture molecule that binds the affinity label, thereby eliminating duplexes that comprise the affinity-labeled DNA.

The present invention also includes methods for eliminating an amplified nucleic acid molecule having a nucleotide sequence that is mutated, compared with the target nucleotide sequence used to produce the amplified nucleic acid molecule, comprising: (a) obtaining a mixture of duplexes of the amplified DNA molecules, wherein the duplex mixture comprises homoduplexes and heteroduplexes, (b) treating the duplex mixture with potassium permanganate, tetraethylammonium chloride, and hydroxylamide to create carbonyl groups in mismatched nucleotides, (c) labeling the carbonyl groups of the duplex mixture with hydrazine derivatized with an affinity label, and (d) incubating the labeled duplex mixture of (c) with an affinity label capture molecule that binds the affinity label, thereby eliminating duplexes that comprise the affinity-labeled DNA. Suitable hydrazine compounds derivatized with an affinity label include 6-((6((biotinoyl)amino)hexanoyl)amino) hexanoic acid hydrazide, N-(aminooxyacetyl)-N′-(D-biotinyl)hydrazine, and L-lysine-N6-[5-hexahydro-2-oxo-1H-thieno[3,4-d]imidazol-4oxy)-1-oxopentyl]-hydrazide.

The present invention further provides methods for eliminating an amplified nucleic acid molecule having a nucleotide sequence that is mutated, compared with the target nucleotide sequence used to produce the amplified nucleic acid molecule, comprising: (a) obtaining a mixture of duplexes of the amplified DNA molecules, wherein the duplex mixture comprises homoduplexes and heteroduplexes, (b) treating the duplex mixture with 1-cyclohexyl-3-{2-[4-(4-methyl)morpholinyl]ethyl}carbodiimide derivatized with an affinity label, and (c) incubating the treated duplex mixture of (c) with an affinity label capture molecule that binds the affinity label, thereby eliminating duplexes that comprise the affinity-labeled DNA. These methods can be performed, for example, with carbodiimide compounds in which at least one of the carbodiimide cyclohexyl groups is replaced with biotin, or in which at least one of the carbodiimide 4-methyl morpholinyl groups is replaced with biotin.

In the methods described herein, amplified nucleic acid molecules can be obtained using the polymerase chain reaction, other amplification methods described herein, or other amplification methods that are standard for those skilled in the art. These methods can be performed with duplexes of DNA, RNA, or DNA-RNA duplexes. Suitable single-strand specific nucleases include S1 nuclease, Mung Bean endonuclease, T4 endonuclease VII, T7 endonuclease I, and CEL I. Suitable DNA polymerases are enzymes with 5′-3′ polymerase and 5′-3′ exonuclease activities, such as E. coli DNA polymerase I, or Taq DNA polymerase. Illustrative affinity labels include biotin, 2-iminobiotin, digoxigenin, fluorescein, coumarin, rhodamine, dinitrophenyl, and the like. Exemplary affinity label capture molecules include avidin, streptavidin, anti-digoxigenin antibody, anti-fluorescein antibody, anti-coumarin antibody, anti-rhodamine antibody, anti-dintrophenyl antibody, and the like. As described above, affinity label capture molecules can be bound to a solid support for convenient separation of labeled heteroduplexes and homoduplexes. Suitable solid supports include cross-linked dextran, agarose, polystyrene beads, silica, silica gel, polyvinyl chloride, polystyrene, cross-linked polyacrylamide, magnetic beads, nitrocellulose- or nylon-based webs, or tubes, plates or the wells of a microtiter plate, such as those made from polystyrene or polyvinylchloride, and the like.

Alternatively, the methods described above can be performed by incorporating an affinity label capture molecule within a heteroduplex, and binding such a heteroduplex with an affinity label. In this approach, the affinity label can be bound to a solid support.

These and other aspects of the invention will become evident upon reference to the following detailed description. In addition, various references are identified below and are incorporated by reference in their entirety.

2. Definitions

In the description that follows, a number of terms are used extensively. The following definitions are provided to facilitate understanding of the invention.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single-stranded or double-stranded.

The term “complement of a nucleic acid molecule” refers to a nucleic acid molecule having a complementary nucleotide sequence and reverse orientation as compared to a reference nucleotide sequence. For example, the sequence 5′ ATGCACGGG 3′ is complementary to 5′ CCCGTGCAT 3′.

The term “contig” denotes a nucleic acid molecule that has a contiguous stretch of identical or complementary sequence to another nucleic acid molecule. Contiguous sequences are said to “overlap” a given stretch of a nucleic acid molecule either in their entirety or along a partial stretch of the nucleic acid molecule.

The term “structural gene” refers to a nucleic acid molecule that is transcribed into messenger RNA (mRNA), which is then translated into a sequence of amino acids characteristic of a specific polypeptide. A “gene of interest” can be a structural gene.

In the context of a double-stranded DNA molecule comprising a gene, the term “upstream” refers to the direction that is toward the 5′-end of the DNA strand (the “antisense strand”) complementary to the strand (the “sense strand”) that serves as the template for transcription, whereas the term “downstream” refers to the opposite direction. As used herein, the terms “upstream” and “5′-ward” are used interchangeably, as are the terms “downstream” and “3′-ward.”

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

As used herein, the term “target nucleotide sequence” refers to a particular nucleotide sequence of interest, which is to be amplified. A “target nucleic acid molecule,” which comprises a target nucleotide sequence, can exist in the presence of other nucleic acid molecules or within a larger nucleic acid molecule. Target nucleic acid molecules can be RNA or DNA.

An “amplicon” is a double-stranded nucleic acid molecule synthesized from a template that is a DNA target nucleotide sequence, or an RNA target nucleotide sequence. If no error in nucleotide sequence is introduced during synthesis, then an amplicon is a double-stranded nucleic acid molecule that is a copy of an original DNA target nucleotide sequence, or that comprises a strand that is a copy of an RNA target nucleotide sequence. An amplicon synthesized with the polymerase chain reaction is a “PCR amplicon.”

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules with free 5′ and 3′ ends. Linear DNA can be prepared from closed circular DNA molecules, such as plasmids, by enzymatic digestion or physical disruption.

A double-stranded nucleic acid molecule is referred to as a “nucleic acid molecule duplex.” A nucleic acid molecule duplex can consist of two strands of DNA, two strands of RNA, or one strand of DNA and one strand of RNA. For example, a “DNA duplex” is double-stranded DNA. When the base sequence of one strand is entirely complimentary to the base sequence of the other strand, then the duplex is a “homoduplex.” In contrast, when a duplex contains at least one base pair, which is not complimentary, then the duplex is called a “heteroduplex.” As an illustration, the products of DNA amplification can form heteroduplexes when amplified DNA molecules differ from the target DNA molecule due to single base substitutions, sequence insertions, sequence deletions, sequence inversions, sequence rearrangements, chimerism, or the like.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “cloning vector” is a nucleic acid molecule, such as a plasmid, cosmid, or bacteriophage, which has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector.

“Integrative transformants” are recombinant host cells, in which heterologous DNA has become integrated into the genomic DNA of the cells.

The term “expression” refers to the biosynthesis of a gene product. For example, in the case of a structural gene, expression involves transcription of the structural gene into mRNA and the translation of mRNA into one or more polypeptides.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, and the like. Where subsequent dissociation of the complement/anti-complement pair is desirable, the complement/anti-complement pair can have a binding affinity of less than 10⁹ M⁻¹. Alternatively, a complement/anti-complement pair can consist of non-identical moieties that form a covalently-associated pair under appropriate conditions (e.g., photo crosslinking).

In the context of the present invention, the members of a complement/anti-complement pair can be an “affinity label” and an “affinity label capture molecule.” As an example, in the complement/anti-complement pair of biotin/avidin (or streptavidin), biotin is an affinity label, whereas avidin (or streptavidin) is an affinity label capture molecule. Methods for using biotin as an affinity label of a PCR product are known in the art (see, for example, Suomatainen and Syvanen, Methods Mol. Biol. 65:67 (1996); Xu et al., Mol. Biotechnol. 17:183 (2001)). Additional examples of affinity labels/affinity label capture molecules include 2-iminobiotin/avidin (or streptavidin), digoxigenin/anti-digoxigenin antibody, fluorescein/anti-fluorescein antibody, coumarin/anti-coumarin antibody, rhodamine/anti-rhodamine antibody, dinitrophenyl/anti-dintrophenyl antibody, and other hapten/anti-hapten antibody pairs (see, for example, Hahn et al., Anal. Biochem. 229:236 (1995); McCreery, Mol. Biotechnol. 7:121 (1997); Wu et al., Methods in Gene Biotechnology (CRC Press 1997); Andreadis and Chrisey, Nucleic Acids Res. 28:5e (2000); Stull, The Scientist 15:20 (2001)).

Due to the imprecision of standard analytical methods, molecular weights and lengths of polymers are understood to be approximate values. When such a value is expressed as “about” X or “approximately” X, the stated value of X will be understood to be accurate to ±10%.

3. Methods for Eliminating Mutated Nucleic Acid Molecules from a Mixture of Amplified Nucleic Acid Molecules

The present invention provides methods for eliminating double-stranded nucleic acid molecules that comprise a mutated nucleic acid molecule strand, wherein the double-stranded nucleic acid molecules have been amplified from a target nucleic acid molecule. Techniques for amplifying a target nucleic acid molecule are well-known to those of skill in the art. The polymerase chain reaction (PCR) is the most widely used method for amplifying a target DNA molecule. Standard techniques for performing PCR are well-known (see, generally, Mathew (Ed.), Protocols in Human Molecular Genetics (Humana Press, Inc. 1991), White (Ed.), PCR Protocols: Current Methods and Applications (Humana Press, Inc. 1993), Cotter (Ed.), Molecular Diagnosis of Cancer (Humana Press, Inc. 1996), Hanausek and Walaszek (Eds.), Tumor Marker Protocols (Humana Press, Inc. 1998), Lo (Ed.), Clinical Applications of PCR (Humana Press, Inc. 1998), Meltzer (Ed.), PCR in Bioanalysis (Humana Press, Inc. 1998), Kochanowski and Reischl (Eds.), Quantitative PCR Protocols (Humana Press, Inc. 1999), and Rapley (Ed.), The Nucleic Acids Protocol Handbook (Humana Press, Inc. 2000)).

A variety of other suitable methods for producing either amplified DNA or RNA molecules are known to those of skill in the art, such as nucleic acid sequence-based amplification, reverse transcriptase-PCR, self-sustained sequence amplification, ligase chain reaction, polymerase/ligase chain reaction, boomerang DNA amplification, rolling circle amplification, restriction amplification, transcription-mediated amplification, strand displacement amplification, and the like (see, for example, Fahy et al., PCR Methods and Applications 1:25 (1991); Walker et al., Proc. Nat'l Acad. Sci. USA 89:392 (1992); Sooknanan and Malek, Biotechnology 13:563 (1995); Finckh et al. (Eds.), Methods in DNA Amplification (Kluwer Academic/Plenum Publishers 1997); Walker and Rapley, Route Maps in Gene Technology (Blackwell Science 1997); Rapley, The Nucleic Acid Protocols Handbook (Humana Press, Inc. 2000); Schweitzer and Kingsmore, Current Opinion in Biotechnology 12:21 (2001)). The methods described herein can also be applied to eliminate nucleotide sequence artifacts that arise during synthetic oligonucleotide synthesis.

Following nucleic acid amplification, heteroduplex formation is induced by standard methods known to those of skill in the art. Example 1 summarizes several of these techniques.

Rendering Mutated Nucleic Acid Molecules Unsuitable for Cloning

The present invention uses nucleases that cleave mismatch DNA to render the heteroduplex population functionally inactive in cloning or other procedures that are dependent on DNA integrity. In this way, the physical separation of the digested heteroduplex DNA from the undigested homoduplex DNA is not required. Suitable nucleases include: S1 nuclease, Mung Bean endonuclease, T4 endonuclease VII (T4E7), T7 endonuclease I (T7EI ), and CEL I of celery. Preferred nucleases include enzymes such as CEL I, T4E7, and T7E1, which have a high specificity for insertions, deletions and single-base pair substitution mismatches. Digestion with these nucleases is carried under conditions where double-strand cleavage, at mismatched sites within the heteroduplex population, is favored. Typically, these reactions are carried out at 10 to 50-fold enzyme excess compared to the amount required for single-strand digestion.

Cloning the undigested homoduplex population into a suitable vector may be carried out by in vitro recombination (see, for example, Wang, Dis. Markers 16:3 (2000)), or by subsequent digestion with suitable restriction endonuclease to generate cohesive terminal ends for ligation to a vector. Heteroduplex DNA molecules cleaved on both strands at the site of nucleotide mismatch would produce DNA fragments that lack recombination sites or restriction digestion sites on both ends of the molecule. Hence, cleaved heteroduplex DNA molecules cannot be incorporated into plasmid vectors efficiently to produce transformation-competent plasmids for in vitro recombination or ligation. Commercially available in vitro recombination cloning kits include: the Gateway system (Invitrogen; Carlsbad, Calif.), and The Creator system (CLONETECH; Palo Alto, Calif.). The necessary sequences for in vitro recombination or for cohesive end ligation to vector may be incorporated into the termini of the DNA by their incorporation into the primers used for DNA amplification. In the case of DNA duplexes that are in whole or in part assembled from chemically synthesized oligonucleotides, the necessary terminal recombination sequences or the restriction endonuclease sites may be chemically synthesized.

Removing Mutated Nucleic Acid Molecules By Affinity Chromatography

The present invention also provides methods for the specific labeling of heteroduplex DNA with a member of a complementary/anti-complementary pair. Following labeling, the heteroduplex DNA can be removed from a population of homoduplex DNA by affinity chromatography employing the other member of the complementary/anti-complementary pair. Examples of complementary/anti-complementary pairs include a biotin/avidin pair, epitope/antibody pair, a ligand/receptor pair, with a biotin/avidin pair preferred. To facilitate removal by affinity chromatography, one member of the complementary/anti-complementary pair can be immobilized on a solid phase matrix, such as a magnetic bead. Useful solid phase supports are well known in the art. Such materials include the cross-linked dextran available under the trademark SEPHADEX; agarose; polystyrene beads (e.g., polystyrene beads about one micron to about five millimeters in diameter); silica; silica gel; polyvinyl chloride, polystyrene, cross-linked polyacrylamide, nitrocellulose- or nylon-based webs such as sheets, strips or paddles; or tubes, plates or the wells of a microtiter plate, such as those made from polystyrene or polyvinylchloride, and the like.

In one approach, heteroduplex DNA is labeled at the site of nucleotide mismatch by use of a modification of the nick-translation reaction first described by Kelly et al., J. Biol. Chem 245:39 (1970)). A single-strand specific nuclease is used to generate a single-strand nick at the site of DNA mismatch. In the presence of DNA polymerase and nucleotide triphosphates, new DNA is synthesized from the exposed 3′OH moiety at the nick site and employing the opposite DNA stand as template. Heteroduplex DNA is labeled when the nick-translation reaction is carried out in the presence of a nucleotide analog that has been conjugated with one member of a complementary/anti-complementary pair. A preferred conjugated complementary group is biotin. Other suitable conjugated complementary groups include: digoxigenin, fluorescein, estradiol and other molecules that can be bound by an antibody for capture. Suitable polymerases for the nick translation reaction include: E. coli DNA polymerase I, Taq DNA polymerase, or other DNA polymerase enzymes with 5′-3′ polymerase and 5′-3′ exonuclease activities. Polymerase enzymes with potent 3′-5′ exonuclease activity is not preferred since this activity, if not well controlled, may result in the labeling of DNA termini of both hetero- and homoduplexes. Suitable nucleases for the generation of single-strand cut at the site of DNA mismatch include: S1 nuclease, Mung Bean endonuclease, T4 endonuclease VII (T4E7), T7 endonuclease I (T7EI ), and CEL I of celery. Among these enzymes, CEL I is preferred due to its high specificity for insertions, deletions, and single-base pair substitution mismatches and its neutral pH optimal for activity, which is compatible with the activity of the polymerases employed for the nick-translation reaction (Oleykowski et al., Nucl. Acids Res. 26:4597 (1998); Yang et al., Biochemistry 39:3533 (2000)). Moreover, CEL I has the advantage in that its activity for making single-strand nicks at the site of nucleotide mismatch is stimulated in the presence of DNA polymerase (Oleykowski et al., Nucl. Acids Res. 26:4597 (1998)).

The present invention also provides methods for the specific labeling of heteroduplex DNA with a member of a complementary/anti-complementary pair at sites of chemical modification of mismatched nucleotides. In the original Chemical Mismatch Cleavage (CMC) protocols employed for mutation analysis, chemically modified mismatch bases were treated with piperdine at 95° C., pH 12, to promote strand cleavage at the site of DNA modification (Cotton et al, Mutation Research 285:125 (1993); Rubin and Schmid, Nucl. Acids Res. 8:4613 (1980); Gogos et al., Nucl. Acids Res. 18:6807 (1990); Lambrinakos et al., Nucl. Acids Res. 27:1866 (1999)). The present invention provides modifications of the CMC procedure to enable labeling of mismatched nucleotides in heteroduplex DNA in order to effect removal by heteroduplex DNA by affinity chromatography.

In brief, following the formation of heteroduplex, mismatched nucleotides are modified with potassium permanganate, tetraethylammonium chloride and hydroxylamide as described by Roberts et al., Nucl. Acids Res. 25:3377 (1997), and by Lambrinakos et al., Nucl. Acids Res. 27:1866 (1999). The newly created carbonyl groups in the modified bases are then labeled with hydrazine derivatized with a member of a complementary/anti-complementary pair. Illustrative complementary/anti-complementary pairs include a biotin/avidin pair, epitope/antibody pair, a ligand/receptor pair, with a biotin/avidin pair preferred. Suitable biotin hydrazine derivatives include those commercially available from, for example, Molecular Probes, Inc. (Eugene, Oreg.), such as: 6-((6((biotinoyl)amino)hexanoyl)amino) hexanoic acid hydrazide, N-(aminooxyacetyl)-N′-(D-biotinyl)hydrazine, and L-lysine-N6-[5-hexahydro-2-oxo-1H-thieno[3,4-d]imidazol-4oxy)-1-oxopentyl]-hydrazide. The biotin-labeled heteroduplex fraction is removed from the homoduplex population by affinity chromatography employing immobilized avidin. Affinity chromatography may be performed using procedures familiar to practitioners skilled in the art, including the use of avidin magnetic beads, or other solid phase matrix as described above.

An alternative method for labeling nucleotide mismatches makes use of reagents such as 1-cyclohexyl-3-{2-[4-(4-methyl)morpholinyl]ethyl}carbodiimide. This reagent was shown to react specifically with mismatched thymine and guanosine bases (Metz and Brown, Biochemistry 8:2312 (1969)) and was used to diagnose single base-pair mismatches in DNA (Novack et al., Proc. Nat'l Acad. Sci. 83:586 (1986)). While Novack et al., Proc. Nat'l Acad. Sci. 83:586 (1986), have mentioned that mismatched DNA tagged with carbodiimide can be purified in principle, they neither described a method for this process nor did they suggest the use of carbodiimide as an affinity labeling reagent to eliminate a DNA population with unwanted nucleotide alterations.

Another aspect of the present invention makes use of 1-cyclohexyl-3-{2-[4-(4-methyl)morpholinyl]ethyl }carbodiimide derivatives to tag DNA containing mutations or artifacts during reverse-transcription or during DNA amplification by PCR. In brief, following the formation of heteroduplex, mismatched nucleotides are labeled with of 1-cyclohexyl-3-{2-[4-(4-methyl)morpholinyl]ethyl}carbodiimide, which has been derivatized with a member of a complementary/anti-complementary pair. The complementary/anti-complementary pair is selected from a group consisting of a biotin/avidin pair, epitope/antibody pair, a ligand/receptor pair, with a biotin/avidin pair preferred. Suitable derivatives include those with replacement of the cyclohexyl- or the 4-methyl morpholinyl groups with biotin. The biotin labeled heteroduplex fraction of the DNA are removed from the homoduplex population by affinity chromatography employing immobilized avidin. Affinity chromatography may be carried employing procedure familiar to practitioners skilled in the art, including the use of avidin magnetic beads, or other solid phase matrix as described above.

The present invention also contemplates kits for performing the selection methods described herein. Such kits can comprise: (1) at least one type of single-strand specific nuclease, such as S1 nuclease, Mung Bean endonuclease, T4 endonuclease VII, T7 endonuclease I, or CEL I; (2) an affinity label, such as biotin, 2-iminobiotin, digoxigenin, fluorescein, coumarin, rhodamine, or dinitrophenyl; (3) an affinity label capture molecule, such as avidin, streptavidin, anti-digoxigenin antibody, anti-fluorescein antibody, anti-coumarin antibody, anti-rhodamine antibody, or anti-dintrophenyl antibody. The affinity label capture molecule can be bound to a solid support such as a magnetic bead. The kits can further comprise components such as a column to purify PCR products, and a polymerase, such as a polymerase with 5′-3′ polymerase and 5′-3′ exonuclease activities (e.g., E. coli DNA polymerase I, or Taq DNA polymerase). A kit may contain all of the additional elements necessary to carry out the technique of the invention, such as buffers, extraction reagents, nucleoside triphosphates, and other consumables of the like.

As an illustration, such a kit can contain all the necessary elements to perform a nucleic acid diagnostic assay described above. A kit may comprise a carrier being compartmentalized to receive in close confinement therein one or more containers, such as tubes or vials. One of the containers may contain a single-strand specific nuclease, and another container may contain a polymerase with 5′-3′ polymerase and 5′-3′ exonuclease activities. A third container may contain an affinity label, such as a biotin dNTP mixture, and a fourth container may contain an affinity label capture molecule. The affinity label capture molecule may be bound to a solid support, such as avidin or streptavidin bound to a magnetic bead. The kit may also include a column to purify DNA products of a polymerase chain reaction. Enzymes and other reagents may be present in lyophilized form or in an appropriate buffer as necessary. A kit may also comprise a means for conveying to the user that kit is employed to eliminate unwanted nucleic acid molecules from a mixture of amplified nucleic acid molecules. For example, written instructions may state that the enclosed components can be used to eliminate heteroduplexes from a mixture of homoduplexes and heteroduplexes. The written material can be applied directly to a container, or the written material can be provided in the form of a packaging insert.

The present invention, thus generally described, will be understood more readily by reference to the following examples, which are provided by way of illustration and are not intended to be limiting of the present invention.

### EXAMPLE 1

### Production of Heteroduplex DNA

PCR primers, nucleotide triphosphates, and polymerase enzyme were removed from amplified DNA products by either gel filtration chromatography employing CHROMA SPIN columns (CLONTECH Laboratories, Inc.; Palo Alto, Calif.) or by affinity chromatography employing QIAQUICK columns (QIAGEN Inc.; Valencia, Calif.). Purified DNA preparations were adjusted to 5 mM Tris-HCl, 5 mM sodium acetate, 2.5 mM EDTA, pH 7.5. Twenty-five microliters of purified DNA (about 50 to 100 ng) were denatured at 96° C. for 5 minutes, and renatured at 72° C. for 60 minutes in a thermocycler instrument.

Those skilled in the art know that other conditions for inducing heteroduplex formation are possible. For example, Qiu et al., Applied and Envir. Microbiol. 67:880 (2001), carried out DNA denaturation at 95° C. for 5 minutes and renaturation at 25° C. for 40 minutes. Lambrinakos et al., Nucl. Acids Res. 27:1866 (1999), described heteroduplex formation in a mixture consisting of 200 ng of DNA in a 40 μl volume containing 0.6 M NaCl, 7 mM MgCl₂, 6 mM Tris-HCl (pH 7.5). The DNA was denatured by boiling for 5 minutes, annealed for 80 minutes at 65° C., and allowed to cool slowly to room temperature overnight. Oleykowski et al., Nucl. Acids Res. 26:4597 (1998), described the preparation of heteroduplex DNA from 50-100 ng of DNA by heating to 94° C. for 1 min followed by slow cooling to room temperature in a buffer containing 20 mM Tris-HCl (pH 7.4), 25 mM KCl, and 10 mM MgCl₂. Other workers have employed a steep temperature gradient for the renaturation step. As an illustration, following a 5 minute denaturation step at 95° C., Howard et al., BioTechniques 27:18 (1999), reanealed the DNA stands in a temperature gradient that spanned from 95° C. to 65° C. over a period of 5 minutes.

### EXAMPLE 2

### Digestion of Heteroduplex DNA with Mismatch-Specific Endonuclease

Conditions for double-strand cleavage of heteroduplex DNA at sites of base-pair mismatch using S1 nuclease were described by Howard et al., BioTechniques 27:18 (1999). Digestion was carried out in an 8 μl reaction volume that contained heteroduplex DNA, 50 mM sodium acetate (pH 4.5), 8 mM NaCl, 1 mM ZnSO₄, 0.5% glycerol, and 0.5 μl of a 1:20 dilution (25 units) of S1 nuclease (Life Technologies Inc.; Rockville, Md.) in a dilution buffer supplied by the manufacturer. Reactions were incubated for 30 minutes at 37° C. and terminated by extraction with phenol/chloroform and precipitation in the presence of 0.3 M sodium acetate (pH 7) and 2.5 volumes of ethanol.

Conditions for double-strand cleavage of heteroduplex DNA at sites of base-pair mismatch using T4 endonuclease VII (T4E7) or T7 endonuclease I (T7E1) were described by Marshal et al., Nature Genetics 9:177 (1995). Digestions were carried out in a 10 μl reaction volume that contained heteroduplex DNA, 50 mM Tris-HCl (pH 8.0), 50 mM potassium glutamate, 10 mM MgCl₂, 5 mM dithiothreitol, 5% glycerol, and suitable amounts of T4E7 or T7E1 endonuclease. Reactions were incubated at 37° C. for 30 minutes to 1 hour, and terminated by extraction with phenol/chloroform and precipitation in the presence of 0.3 M sodium acetate (pH 7) and 2.5 volumes of ethanol. T4E7and T7E1 are commercially available from Amersham Pharmacia Biotech Inc. (Piscataway, N.J.) and New England Biolabs Inc. (Beverly, Mass.), respectively. Optimal amounts of endonuclease and digestion time to effect single- and double-stranded digestion at the site of nucleotide mismatch can be determined using a test heteroduplex with defined sets of mismatched bases. Typically, between 0.5 to 2.5 units of enzyme are sufficient to digest one DNA strand at the site of mismatch in a one-hour incubation at 37° C. Double-strand digestion at mismatched sites may require 10- to 50-fold greater enzyme than that required for single stand digestion.

Conditions for cleavage of heteroduplex DNA at sites of base-pair mismatch using CEL I endonuclease was described by Oleykowski et al., Nucl. Acids Res. 26:4597 (1998). Fifty to 100 ng of heteroduplex DNA were digested in a 20 μl volume containing 20 mM Tris-HCl (pH 7.4), 25 mM KCl, 10 mM MgCl₂, 0.5 unit Taq polymerase (Perkin Elmer), and 100 ng purified CEL I (about 0.2 units). Digestion was carried out at 45° C. for 30 minutes and stopped by the addition of o-phenanthroline to 1 mM final concentration, and incubated for an additional 10 minutes at 45° C. An approximate 50-fold excess of CEL I is generally required for double-strand cleavage at mismatched sites.

### EXAMPLE 3

### Specific Labeling of Heteroduplex DNA with Biotin by Nick-translation

Heteroduplex DNA is labeled with biotin from the site of nucleotide mismatch by use of a modification of the nick-translation reaction first described by Kelly et al., J. Biol. Chem 245:39 (1970). Heteroduplex DNA is digested with a mismatch-specific endonuclease to create single-strand break at the site of DNA mismatch. A number of mismatch-specific endonucleases are suitable for this application. For the present invention, the use of T4E7, T7E1, or CEL I is convenient, because the neutral pH optima and the buffer requirements for activity of these endonucleases are comparable with those of the polymerase enzymes employed for the subsequent polymerase reaction. Accordingly, buffer exchange between the nicking reaction and the polymerase reaction is not necessary and both reactions may in fact be carried out simultaneously.

Digestion conditions are similar to those described by Marshal et al., Nature Genetics 9:177 (1995). Briefly, 50 to 100 ng of heteroduplex DNA are digested with T4 endonuclease VII (T4E7) or T7 endonuclease I (T7E1) to generate single-strand cuts at the sites of DNA mismatch. The optimal amount of nuclease can be determined using a model heteroduplex. Digestions can be carried out in a 20 μl reaction volume consisting of heteroduplex DNA, 50 mM Tris-HCl (pH 8.0), 50 mM potassium glutamate, 10 mM MgCl₂, 5 mM dithiothreitol, 5% glycerol, and 0.5 to 2.5 units of T4E7(Amersham Pharmacia Biotech Inc., Piscataway, N.J.) or T7E1 (New England Biolabs Inc, Beverly, Mass.). Reactions are incubated at 37° C. for 30 minutes after which the reaction is transfer to ice until ready for the polymerase reaction.

Conditions for single-strand cleavage of heteroduplex DNA at sites of base-pair mismatch using CEL I endonuclease is similar to that described by Oleykowski et al., Nucl. Acids Res. 26:4597 (1998). The optimal amount of CEL I employed may need to be determined using a model heteroduplex. In general, 50 to 100 ng of heteroduplex DNA are digested in a 20 μl volume containing 20 mM Tris-HCl (pH 7.4), 25 mM KCl, 10 mM MgCl₂, 0.5 unit Taq polymerase (Perkin Elmer) and 100 to 200 ng purified CEL I (about 0.2 to 0.4 units). Digestion is carried out at 45° C. for 30 minutes and stopped by the addition of o-phenanthroline to 1 mM final concentration and incubated for an additional 10 minutes at 45° C. The reaction is then transferred to ice.

Biotin labeling of the heteroduplex can be out in a 30 μl nick translation reaction containing 20 μl of either the CEL I, T4E7, or T7E1 digested heteroduplux DNA, 1 μl of 10× polymerase buffer (0.5 M Tris.HCl (pH 7.5), 0.1 MgCl₂, 1 mM dithiothreitol, 500 μg/ml bovine serum albumin), 25 nmoles of dCTP, dTTP and dGTP, 2.5 nmoles dATP, 22.5 nmoles biotin-7-dATP, and 2.5 units E. coli polymerase I. The nick translation reaction is carried at 16° C. for 60 minutes. Unincorporated nucleotides are removed by gel-filtration employing a CHROMA SPIN column (CLONETECH) or by affinity chromatography employing a QIAQUICK column (QIAGEN).

Since T4E7, T7E1, and CEL I are active under conditions where polymerase used for the nick translation reaction is also active, it is possible to carry out the nicking reaction and the polymerase reaction simultaneously. The reactions are carried out in a 30 μl volume consisting of heteroduplux DNA, 50 mM Tris-HCl (pH 8.0), 50 mM potassium glutamate, 10 mM MgCl₂, 5 mM dithiothreitol, 5% glycerol, and 0.5 to 2.5 units of T4E7(Amersham Pharmacia Biotech Inc., Piscataway, N.J.) or T7E1 (New England Biolabs Inc, Beverly, Mass.) or about 0.2 to 0.4 units CEL I, 25 nmoles of dCTP, dTTP and dGTP, 2.5 nmoles dATP, 22.5 nmoles biotin-7-dATP, and 2.5 units E. coli polymerase I. The reactions are incubated at 37° C. for 30 minutes and terminated by the addition of EDTA to 5 mM and transfer to ice.

Since CEL I is active at high temperature, it is possible to label heteroduplex with biotin by simultaneously performing nicking and polymerase reactions at 65° C. The reaction is carried out in a 30 μl volume consisting of heteroduplex DNA, 20 mM Tris-HCl (pH 7.4), 25 mM KCl, 2 mM MgCl₂, 25 nmoles of dCTP, dTTP and dGTP, 2.5 nmoles dATP, 22.5 nmoles biotin-7-dATP, 100 ng purified CEL I (about 0.2 units), and 0.5 unit Taq polymerase (Perkin Elmer). The reaction is incubated at 65° C. for 10 minutes and is stopped by the addition of o-phenanthroline to 1 mM and EDTA to 10 mM final concentration. The high temperature improves the efficiency of digestion at single base-pair mismatches by destabilizing adjacent bases. However, at this temperature, it is important to stabilize the DNA termini from “breathing” to prevent their digestion and subsequent labeling with biotin. This objective is best achieved by the inclusion of a short GC-rich sequence at the 5′ termini of the PCR primers used to generate the DNA. The absence of thymine nucleotides within the 5′ most 12 base of the primers would also inhibit the inadvertent incorporation of biotin-7-dATP at the DNA termini.

### EXAMPLE 4

### Affinity Labeling of Mismatched Nucleotides Chemical Modified by Potassium Permanganate, Tetraethylammonium Chloride and Hydroxylamide Chloride

Modification of mismatches bases with potassium permanganate, tetrethylammonium chloride, and hydroxylamide chloride can be accomplished in a single tube reaction similar that that described by Lambrinakos et al., Nucl. Acids Res. 27:1866 (1999). Briefly, 100 to 200 ng of heteroduplex DNA are incubated in 20 μl of 1 mM potassium permanganate and 3 M tetrethylammonium chloride for 5 minutes at 25° C. An equal volume of 11.5 M hydroxylamide chloride (adjusted to pH 6.0 with diethylamine) is added and incubated for 40 minutes at 25° C. The modified DNA is precipitated with 2.5 volumes of ethanol in the presence of 0.3 M sodium acetate, pH 8. The reactive carbonyl group generated at the modified base is labeled with biotin using the aldehyde reaction agent, N′-aminoxymethylcarbonylhydrazino D-biotin (Ide et al., Biochemistry 31:8276 (1993)). One hundred to 200 ng of modified DNA in 50 μl of phosphate buffer (20 mM, pH 7) are added to 50 μl of a 5 mM aqueous solution of N′-aminoxymethylcarbonylhydrazino D-biotin and allowed to react at 37° C. for 30 minutes. Unreacted N′-aminoxymethylcarbonylhydrazino D-biotin is removed from the labeled heteroduplex DNA by gel-filtration employing a CHROMA SPIN column (CLONETECH) or by affinity chromatography employing a QIAQUICK column (QIAGEN).

### EXAMPLE 5

### Affinity Labeling of Mismatched Nucleotides by of 1-cyclohexyl 3-{2-[4-(4-methyl)morpholinyl]ethyl}carbodiimide Derivatives

In brief, following the formation of heteroduplex, mismatched nucleotides are labeled with of 1-cyclohexyl-3-{2-[4-(4-methyl)morpholinyl]ethyl }carbodiimide, which has been derivatized with biotin (Biotin CDI). Suitable derivatives include those with replacement of the cyclohexyl- or the 4-methyl morpholinyl groups with D-biotin. The labeling reaction is carried in 50 μl volume containing 20 to 200 ng of heteroduplex DNA, 100 nM sodium borate (pH 8.0) and 20 mM of biotin CDI. The reaction was incubated at 30° C. for 3 hours. Unreacted biotin CDI is removed from the labeled heteroduplex DNA by gel-filtration employing a CHROMA SPIN column (CLONETECH) or by affinity chromatography employing a QIAquick column (QIAGEN).

### EXAMPLE 6

### Affinity Selection of Mismatched DNA

Following the removal of unincorporated nucleotides, biotin labeled heteroduplex DNA is removed from the unlabeled homoduplex population by affinity chromatography with the use of streptavidin magnetic beads (e.g., DYNABEADS Streptavidin; Dynal Biotech Inc.; Lake Success, N.Y.). Affinity chromatography is carried out in a 100 μl reaction volume containing DNA, 1 M NaCl, 10 mM Tris.HCl (pH 7.5), 1 mM EDTA and prewashed DYNABEADS Streptavidin (1 mg beads/40 pmoles of input DNA). The sample is incubated at 43° C. for 1 hour under constant agitation after which the DYNABEADS Streptavidin along with the biotin labeled heteroduplex DNA are removed with a magnetic field. The remaining DNA is desalted by gel-filtration or ethanol precipitation and is ready for insertion into suitable plasmid vector or other downstream applications.

### EXAMPLE 7

### Cloning of Homoduplex DNA

Homoduplex DNA may be digested in suitable restriction endonuclease to create commentary ends for ligation into suitable vectors. Alternatively, homoduplex DNA is mobilized into plasmid vectors using in vitro recombination kits in accordance to the direction of the vendors (Invitrogen, Carlsbad, Calif.; Clontech, Palo Alto, Calif.).

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for eliminating an amplified nucleic acid molecule having a nucleotide sequence that is mutated, compared with the target nucleotide sequence used to produce the amplified nucleic acid molecule, comprising,
(a) obtaining a mixture of duplexes of amplified nucleic acid molecules, wherein the duplex mixture comprises homoduplexes and heteroduplexes,
(b) treating the duplex mixture with a single-strand specific nuclease that cleaves a mismatched site within a heteroduplex, and
(c) subcloning the nuclease-treated duplex mixture, wherein a cleaved duplex is unsuitable for subcloning.

2. The method of claim 1, wherein the single-strand specific nuclease is selected from the group consisting of S1 nuclease, Mung Bean endonuclease, T4 endonuclease VII, T7 endonuclease I, and CEL I.

3. The method of claim 1, wherein the nucleic acid molecule is DNA.

4. The method of claim 1, wherein the amplified nucleic acid molecules are obtained by using the polymerase chain reaction.

5. A method for eliminating an amplified nucleic acid molecule having a nucleotide sequence that is mutated, compared with the target nucleotide sequence used to produce the amplified nucleic acid molecule, comprising,
(a) obtaining a mixture of duplexes of the amplified DNA molecules, wherein the duplex mixture comprises homoduplexes and heteroduplexes,
(b) cleaving the duplex mixture with a single-strand specific nuclease that cleaves a mismatched site within a heteroduplex, wherein the cleavage produces an exposed 3′OH moiety,
(c) treating the cleaved duplex mixture with a DNA polymerase and nucleotide analog conjugated with an affinity label to synthesize DNA that comprises the affinity label, and
(d) incubating the treated duplex mixture of (c) with an affinity label capture molecule that binds the affinity label, thereby eliminating duplexes that comprise the affinity-labeled DNA.

6. The method of claim 5, wherein the single-strand specific nuclease is selected from the group consisting of S1 nuclease, Mung Bean endonuclease, T4 endonuclease VII, T7 endonuclease I, and CEL I.

7. The method of claim 5, wherein the DNA polymerase is an enzyme with 5′-3′ polymerase and 5′-3′ exonuclease activities.

8. The method of claim 7, wherein the DNA polymerase is either E. coli DNA polymerase I, or Taq DNA polymerase.

9. The method of claim 5, wherein the amplified nucleic acid molecules are obtained by using the polymerase chain reaction.

10. The method of claim 5, wherein the affinity label is biotin and the affinity label capture molecule is either avidin or streptavidin.

11. The method of claim 5, wherein the affinity label capture molecule is bound to a solid support.

12. The method of claim 11, wherein the solid support is a magnetic bead.

13. A method for eliminating an amplified nucleic acid molecule having a nucleotide sequence that is mutated, compared with the target nucleotide sequence used to produce the amplified nucleic acid molecule, comprising,
(a) obtaining a mixture of duplexes of the amplified DNA molecules, wherein the duplex mixture comprises homoduplexes and heteroduplexes,
(b) treating the duplex mixture with potassium permanganate, tetraethylammonium chloride, and hydroxylamide to create carbonyl groups in mismatched nucleotides,
(c) labeling the carbonyl groups of the duplex mixture with hydrazine derivatized with an affinity label, and
(d) incubating the labeled duplex mixture of (c) with an affinity label capture molecule that binds the affinity label, thereby eliminating duplexes that comprise the affinity-labeled DNA.

14. The method of claim 13, wherein the amplified nucleic acid molecules are obtained by using the polymerase chain reaction.

15. The method of claim 13, wherein the affinity label is biotin and the affinity label capture molecule comprises either avidin or streptavidin.

16. The method of claim 13, wherein the hydrazine derivatized with biotin is selected from the group consisting of 6-((6((biotinoyl)amino)hexanoyl)amino) hexanoic acid hydrazide, N-(aminooxyacetyl)-N′-(D-biotinyl)hydrazine, and L-lysine-N6-[5-hexahydro-2-oxo-1H-thieno[3,4-d]imidazol-4oxy)-1-oxopentyl]-hydrazide.

17. The method of claim 13, wherein the affinity label capture molecule is bound to a solid support.

18. The method of claim 17, wherein the solid support is a magnetic bead.

19. A method for eliminating an amplified nucleic acid molecule having a nucleotide sequence that is mutated, compared with the target nucleotide sequence used to produce the amplified nucleic acid molecule, comprising,
(a) obtaining a mixture of duplexes of the amplified DNA molecules, wherein the duplex mixture comprises homoduplexes and heteroduplexes,
(b) treating the duplex mixture with 1-cyclohexyl-3-{2-[4-(4-methyl)morpholinyl]ethyl }carbodiimide derivatized with an affinity label, and
(c) incubating the treated duplex mixture of (c) with an affinity label capture molecule that binds the affinity label, thereby eliminating duplexes that comprise the affinity-labeled DNA.

20. The method of claim 19, wherein the amplified nucleic acid molecules are obtained by using the polymerase chain reaction.

21. The method of claim 19, wherein the affinity label is biotin and the affinity label capture molecule comprises either avidin or streptavidin.

22. The method of claim 21, wherein at least one of the carbodiimide cyclohexyl groups is replaced with biotin.

23. The method of claim 21, wherein at least one of the carbodiimide 4-methyl morpholinyl groups is replaced with biotin.

24. The method of claim 19, wherein the affinity label capture molecule is bound to a solid support.

25. The method of claim 24, wherein the solid support is a magnetic bead.
